(19) United States
(12) Patent Application Publication
(10) Pub. No.: US 2009/0287108 A1
(43) Pub. Date: Nov. 19, 2009

(54) SYSTEM FOR STUDY AND TREATMENT OF BEHAVIOR DYSREGULATION

(76) Inventor: Mark M. Levy, Raanana (IL)

Correspondence Address: David Klein;DEKEL PATENT LTD., Beit HaRof' im, 18 Menuha VeNahala Street, Room 27, REHOVOT, IL

(21) Appl. No.: 12/468,062

(22) Filed: May 19, 2009

Related U.S. Application Data
(60) Provisional application No. 61/054,207, filed on May 19, 2008.

(51) Int. Cl. A61B 5/0476

(52) U.S. Cl. 600/544

(57) ABSTRACT

A method for changing appearance of EEG or a relationship of brain wave patterns while achieving behavioral changes, the method including individualized assessing of dysregulated patterns of brain function using topographic quantitative electroencephalogram (LORETA [low resolution brain electromagnetic tomography] qEEG) or regular EEG recollection, modifying or priming neuroplasticity with transcranial direct current stimulation (tDCS) or with magnetic or electromagnetic fields to modify cortical excitability of neurons in dysregulated areas, and self-regulating LORETA patterns with neurofeedback to change an appearance of EEG or a relationship of brain wave patterns while achieving behavioral changes.

### CROSS-REFERENCE TO RELATED APPLICATIONS

This application claims priority under 35 USC §119 to U.S. Provisional Patent Application Ser. No. 61/054,207, filed May 19, 2008, which is incorporated herein by reference.

### FIELD OF THE INVENTION

The present invention relates generally to the study and treatment of brain dysfunction conditions.

### BACKGROUND OF THE INVENTION

The world is experiencing what has been called an “epidemic of psychiatric disorders”. According to the World Health Organization (WHO), by the year 2020, five of the ten leading causes of morbidity and mortality affecting mankind will be of psychiatric origin (depression, anxiety disorders, drug abuse and smoking, post traumatic stress disorder, and alcohol abuse). In addition, presently 10-15% of children are experiencing what is referred to as the trendy condition of attention deficit and hyperactivity disorder. Amazingly, these brain disorders are not associated with a readily identifiable anatomic brain pathology and are therefore referred to as “functional brain disorders”. Treatments that can impact positively on these functional disorders will greatly benefit humanity and will also create the opportunity for substantial financial gain.

The human brain is an exquisite organ with an almost endless capacity for change. This flexibility is particularly evident in the younger years but evidence from recent studies suggests that adaptations in brain function (plasticity) occur even at an advanced age. Functional disorders of the brain most probably result from interactions between the genetic characteristics we are born with and the environment we have grown and developed in, or the one we currently live in. It is yet unclear how precisely this interaction between gene makeup and environment affects brain function. If we take the example of how we learn new material, there is evidence that new concepts are incorporated through changes in neuronal plasticity, more specifically, the creation of long-term potentiation in specific neuronal networks. Neuronal plasticity refers to the way neurons interact with each other and the way this interaction is translated into biochemical signals within each cell. It is quite probable that alterations in neuronal plasticity lay at the basis of these functional disorders of the brain.

Day to day activities like the monitoring of mood, appetite, impulses, sex drive, and many more, require the normative interaction of many brain areas working in consonance, what has been referred to as brain networks. For example, the occurrence of a significant stressful situation will lead to the simultaneous activation of a number of brain areas like:

1. Amygdala: leads the immediate response of the body to the threat

2. Prefrontal cortex: assesses and modulates whether the initial amygdala response is appropriate or requires activation of other brain functions

3. Prefronto-striatal-hypothalamic projections: leads the somatic adrenocortical response to stress that prepares the body for the approaching or occurring insult

4. Hypothalamic-Pituitary-Adrenal response leads to increases in ACTH (Adrenocorticotropic Hormone) and cortisol secretion

5. Shut down and feedback mechanisms are activated after the threat has subsided.

The processes cited previously include the activation of cascades of cellular mechanisms that are, and have to be, in a state of shaping and reshaping from the moment the person is born to the moment he/she dies. At the level of the isolated neuron, synaptic, cytoplasmatic, and intranuclear processes involve many known and yet to be discovered molecular mechanisms. At the level of neuronal networks, “how neurons talk to each other” or “how neurons learn from each other”, it involves processes referred to as “neuronal plasticity”.

The study of brain plasticity in functional disorders of the brain is at its infancy and its potential use in therapeutics is huge. It is believed that the present invention has the potential of improving the load of many patients suffering from these “functional disorders of the brain”.

As examples of “functional disorders of the brain” we may discuss depression and anxiety. Depression and anxiety are not localized disorders of the brain. There is no “center of depression” in the brain. If, for example, the left dorsolateral prefrontal cortex and the anterior cingular cortex are found to be hypofunctional in a depressed individual, this may explain problems with motivation and concentration. However, it says nothing about why the person is experiencing sleep, appetite, and sexual disturbances. For this we need additional changes in deeper structures like the caudate nucleus, the hypothalamus and more. This speaks to a diffuse dysfunction of how neuronal networks communicate in the brain (“how neurons talk to each other”). Thus, a treatment that is capable of influencing neuroplasticity in the right direction will have a major impact on the “functional disorders of the brain”.

It is easy to foresee situations in which these complex processes that have been shaping and reshaping themselves since the moment of birth can enter a state of dysregulation or lack of control. For example, the psychiatric literature on stress or PTSD (post-traumatic stress disorder) includes many descriptions of stages in which the prefrontal cortex is hypofunctional and the amygdalar cortex is hyperfunctional. This means that in the presence of a stress our brain and our body may respond as if a major catastrophe is approaching while the prefrontal cortex, in charge of modulating this response, is less effective in diminishing this response to a more appropriate level. As a consequence, our body may respond to environmental stresses as “always is a state of war”. This situation is conducive to a chronic state of neuronal activation that eventually leads to illnesses, psychological suffering and more.

### SUMMARY OF THE INVENTION

The present invention seeks to treat brain dysfunction conditions, as is described more in detail hereinbelow. The present invention, through its effects on neuronal plasticity, aims at correcting neurobiological situations in which the synchronization of brain function has gone off target.

There is provided in accordance with an embodiment of the present invention a method for changing appearance of EEG or a relationship of brain wave patterns while achieving behavioral changes, the method including individualized assessing of dysregulated patterns of brain function using topographic quantitative electroencephalogram (LORETA [low resolution brain electromagnetic tomography] qEEG) or regular EEG recollection, modifying or priming neuroplasticity with transcranial direct current stimulation (tDCS), transcranial alternating current stimulation (tACS), transcranial electromagnetic or magnetic stimulation (TMS), transcranial ultrasound wave stimulation (TUSS), radiofrequency or low intensity laser stimulation, to modify cortical excitability of neurons in dysregulated areas, and self-regulating LORETA patterns with neurofeedback to change an appearance of EEG or a relationship of brain wave patterns while achieving behavioral changes.

There is provided in accordance with an embodiment of the present invention a system for changing appearance of EEG or a relationship of brain wave patterns while achieving behavioral changes, the system including a device for individualized assessing of dysregulated patterns of brain function using topographic quantitative electroencephalogram (LORETA qEEG) or regular EEG recollection, a device for modifying or priming neuroplasticity with transcranial direct current stimulation (tDCS), transcranial alternating current stimulation (tACS), transcranial electromagnetic or magnetic stimulation (TMS), transcranial ultrasound wave stimulation (TUSS), radiofrequency or low intensity laser stimulation, to modify cortical excitability of neurons in dysregulated areas, and a device for self-regulating LORETA patterns with neurofeedback to change an appearance of EEG or a relationship of brain wave patterns while achieving behavioral changes.

### DETAILED DESCRIPTION OF EMBODIMENTS

The present system is designed to treat brain dysfunction conditions (excluding those entities with anatomical brain damage), such as:

1. Conditions in which prefrontal cortex has become less effective in the process of regulation of other brain functions. This category includes:

a) Obesity and smoking addiction. Obesity is a disease afflicting the western world. Obesity and a sedentary life style are conducive to many illnesses, e.g., increased blood pressure, dyslipidemias, cardiovascular and cerebrovascular disease, depression and anxiety, and early death. Causes of obesity are numerous and can be genetic, familial, environmental, and more. Smoking is well established as being a major factor in cardiovascular disease, lung cancer, chronic obstructive lung disease, and more. In the present invention, both conditions (obesity and smoking addiction) are situations of lack of control by functions of the prefrontal cortex. In obese individuals the common struggle between the “tasty food” and the need for restraint becomes a battle ground in which the will power loses to the impulse to eat. In cigarette addiction, the wish to quit loses when the impulse to smoke becomes insurmountable. Normal reasoning processes cannot overcome the impulse to eat or smoke. The present invention can “teach” the prefrontal cortex to be more effective in regulating food and cigarette craving.

b) Other addictive substances such as, but not only pharmacological, recreational drugs and alcohol, video games, compulsive need of fire arms use, compulsive shopping, kleptomania, bulimia, uncontrolled sex drive, pedophilia, etc.

2. Conditions in which the regulation of neuronal networks cannot reach homeostasis because the allostatic load (response to stress or insult, either biological or psychological) does not allow the system to be “turned down or reset” to a normal state. Put in simple language, the volume of communication between neuronal networks is either too high or too low and not synchronized. We view these conditions as failures of neuronal plasticity processes.

a) Depressive and anxiety disorders. Many studies suggest that in depression both the prefrontal and the anterior cingular cortex are in a dysfunctional state, particularly hypofunctional. As a consequence their ability to integrate cognitive performance with visceral function (limbic system) disintegrates. Treatments, either pharmacological or somatic, such as electroconvulsive therapy or transcranial magnetic stimulation, which correct this function lead to temporary recovery from depression. The present invention facilitates this process through self-regulation of cortical brain wave patterns

b) Post-traumatic stress disorder (PTSD). Psychological dysfunctional states following severe trauma are very common. Typically a person suffering from PTSD will have intrusive memories of the traumatic events during the day or during sleep and these will be conducive to poor psychosocial functioning, mental suffering, poor occupational record, and more. Many studies of brain function of people suffering from PTSD show dysregulation of brain function with either hypo or hyperfunctioning brain areas when presented with the memory of the trauma. Facilitated primed neurofeedback to the prefrontal cortex, as suggested in this invention, will allow stricter monitoring of these intrusive recollections during both awake and sleep stages.

c) Attention Deficit Hyperactivity Disorder (ADHD). Most of the literature in neurofeedback has been in the area of attention deficit hyperactivity disorder of children; very little has been done in adults. The results are basically positive. However, there are drawbacks. The process of self-regulation requires between 30-40 training sessions; a significant proportion of children cannot learn neurofeedback; and it is unclear how well what is learned during the training sessions can be transferred to real life situations, short and long term. The present invention brings true and permanent changes in the self-regulating capabilities of children and adults suffering from ADHD.

d) Other conditions like insomnia, anorexia, frigidity, etc are also included in this group.

e) Memory dysfunctions in all age group individuals.

The invention includes three aspects:

1. Individualized assessment of dysregulated patterns of brain function using topographic quantitative electroencephalogram (LORETA [low resolution brain electromagnetic tomography] qEEG) or regular EEG recollection continuously or digitally. In some conditions an appropriate stimulus should be used to make evident the appearance of abnormal EEG patterns or unbalance of normal brain waves. This includes the use of virtual reality means, with visual and audio factors that mimic the unwanted situations, as provocative stimuli like foods in obesity, drinks in alcoholism, disruptive scenes in ADHD, war or shocking views in PTSD and others. In addition, stimuli including different odors, flavors, and temperatures and other tactile stimuli or combination of the former also can be used for enhancement of subtle changes in the EEG.

2. Modifications or priming of neuroplasticity with transcranial direct current stimulation (tDCS), transcranial magnetic stimulation (TMS) or electromagnetic stimulation, transcranial alternating current stimulation (tACS), transcraneal ultrasound waves stimulation (TUSS), radio frequency stimulation, low intensity laser stimulation or other fields (increasing or decreasing cortical excitability of neurons) in dysregulated areas. Stimulation of the brain surface or cortex causes stimulation of deeper brain structures by neuronal interconnections without the need of strong stimulus parameters for direct deep stimulation. Also computerized virtual reality sets can be used for stimulation or as an aid together with another stimulation technique.

3. Self-regulation of LORETA or EEG patterns with neurofeedback, including visual, audio or combined neurofeedback programs. Virtual reality settings can also be used for neurofeedback. The neuro-feedback training program is carried out either simultaneously or in an alternating manner while recollecting the EEG patterns produced. Through neurofeedback the individual is able to modify brain wave patterns that in turn impact on brain plasticity. In such programs, additional sessions of stimulation may be required to achieve more sustainable results by the creation of new interneuron connections or synapses and/or synthesis of proteins that may ensure the long term effects.

The individual baseline EEG collected (first step in the process) can be processed and differentiated into categories, based on statistics and/or algorithms, which will define the individual treatment using a fitting protocol of stimulation selected for the specific category and an appropriate mode of neurofeedback training. Different modalities of signal processing may be used including filtering systems including Kalman and others, Fast Fourier Transform and all its variables, fuzzy logic, artificial intelligence systems, error in variable models, stochastic casual systems, network systems including those based on rough set theory and others, to elaborate effective algorithms that may assist in the categorization and segmentation of the groups and individuals to be treated.

The process of EEG collection and categorization can be automatically or manually done, as well as the protocols of stimulation and feedback training. In addition, the neurofeedback training protocol can also be automatically or manually adjusted for further advancement in the training process. The neurofeedback protocols can be specific to the disorder to treat or general to certain dysfunction categories. Following the first interaction with this methodology, further treatment sessions can be done in a personalized individual manner, in a doctor's office setup or at the patient home. Further check-ups done in the doctor's office may need some adjustment of the parameters or finalization of treatment.

One objective of the invention is to achieve behavioral or conduct improvement by affecting the activity of the prefrontal cortex and its interaction and more effective modulation of deeper brain structures like cingular cortex, amygdalar cortex and prefronto-striatal projections involved in the targeted behavior imbalance, reflected by the accomplishment of short and long term changes in the EEG patterns.

The system claimed in this invention includes a multi-channel EEG reception device, a brain stimulation device (DC or AC electrical stimulation device, an electromagnetic or magnetic field stimulation device, ultrasound stimulation device, radiofrequency or low intensity laser device) and a display and appropriate software for neurofeedback training arrangement, where the EEG recollection is free from the stimulation artifacts with suitable algorithms for categorization of baseline EEG and for selection of proper protocol of stimulation and neuron-feedback program with its coordination for best treatment results. Treatment units consist of full size doctor's units and portable patient's units. Online monitoring and updates are also considered as part of the scope of this invention, as well as online sessions and marketing.

The scope of the present invention includes both combinations and subcombinations of the features described hereinabove as well as modifications and variations thereof which would occur to a person of skill in the art upon reading the foregoing description and which are not in the prior art.

What is claimed is:

1. A method for changing appearance of EEG or a relationship of brain wave patterns while achieving behavioral changes, the method comprising:
individualized assessing of dysregulated patterns of brain function using topographic quantitative electroencephalogram (LORETA [low resolution brain electromagnetic tomography] qEEG) or regular EEG recollection;
modifying or priming neuroplasticity with transcranial direct current stimulation (tDCS), transcranial alternating current stimulation (tACS), transcranial electromagnetic or magnetic stimulation (TMS), transcranial ultrasound wave stimulation (TUSS), radiofrequency or low intensity laser stimulation, to modify cortical excitability of neurons in dysregulated areas; and
self-regulating LORETA patterns with neurofeedback to change an appearance of EEG or a relationship of brain wave patterns while achieving behavioral changes.

2. A system for changing appearance of EEG or a relationship of brain wave patterns while achieving behavioral changes, the system comprising:
a device for individualized assessing of dysregulated patterns of brain function using topographic quantitative electroencephalogram (LORETA qEEG) or regular EEG recollection;
a device for modifying or priming neuroplasticity with transcranial direct current stimulation (tDCS), transcranial alternating current stimulation (tACS), transcranial electromagnetic or magnetic stimulation (TMS), transcranial ultrasound wave stimulation (TUSS), radiofrequency or low intensity laser stimulation, to modify cortical excitability of neurons in dysregulated areas; and
a device for self-regulating LORETA patterns with neurofeedback to change an appearance of EEG or a relationship of brain wave patterns while achieving behavioral changes.
